(19) United States
(12) Patent Application Publication
(10) Pub. No.: US 2004/0081990 A1
(43) Pub. Date: Apr. 29, 2004

(54) Method for the identification of essential genes and therapeutic targets

(75) Inventors: Roger C. Levesque, St.-Nicolas (CA); Francois Sanschagrin, Quebec (CA); Guy Cardinal, Quebec (CA)

Correspondence Address: MERCHANT & GOULD PC, P.O. BOX 2903, MINNEAPOLIS, MN, 55402-0903, US

(73) Assignee: UNIVERSITE LAVAL, Quebec (CA)

(21) Appl. No.: 10/647,566

(22) Filed: Aug. 25, 2003

Related U.S. Application Data
(63) Continuation of application No. 09/508,891, filed on Jun. 2, 2000, now abandoned.
Application No. 09/508,891 is a 371 of international application No. PCT/CA98/00893, filed on Sep. 21, 1998.

(30) Foreign Application Priority Data
Sep. 19, 1997 (CA) 2,215,870

(51) Int. Cl. C12Q 1/68; C12P 19/34; C12N 15/85

(52) U.S. Cl. 435/6; 435/455; 435/91.2

(57) ABSTRACT

The present invention relates to a method of identifying essential genes in a genome, based on an insertional mutagenesis of a population of cells or of DNA molecules and subjecting this population of cells or DNA molecules to an amplification process, whereby this total population of cells or DNA molecules which statistically represents at least one full insertionally mutated genome is amplified with at least two primer pairs and the extension products analysed, in order to distinguish essential genes from dispensable genes. The present invention is especially suited to the functional analysis of microbial genomes, and especially to haploid genomes.

### FIELD OF THE INVENTION

The present invention relates to the identification of essential genes in a given genome. More specifically, the invention relates to the identification of essential genes in a diploid organism in which homozygocity conversion is efficient or in a haploid organism. The present invention also relates to the identification of therapeutic targets and more specifically to therapeutic targets in bacteria.

### BACKGROUND OF THE INVENTION

The human genome project as well as genome projects of model organisms have opened the area of genomics. Although thousands of genetic sequences are available in data bases, only a small minority thereof have a recognized function. It has now become evident that biological functions cannot be solely deduced by computer approaches and that even in integrated format, databases present significant limitations.

Large amounts of data, from the partial or complete DNA sequences of microbial genomes are also rapidly accumulating in databases. Genome amplification methods and genotyping methods have been described (see for example Cheung et al., 1996, Proc. Natl. Acad. Sci. USA 93:19676-19679). There is heightened expectations that the increasingly powerful computer analyses will be able to yield biological function from these DNA sequence. However, it is becoming clear that even for microbial genomes, the sole information in databases will not be sufficient to deduce the biological function. Thus, it becomes apparent that whole genome or genome-based analysis of biological function could provide significant results. Indeed, such analysis could be, for example, the next phase in microbial genomics, particularly as it pertains to finding novel therapeutic targets in bacteria.

Expression of a subset of genes is essential for survival of the eukaryotic and prokaryotic cells; mutations in these genes give rise to a lethal phenotype. Recently, the number of lethal loci has been estimated in a number of life forms serving as model organisms for genome projects: Drosophila (3,600 essential genes), Caenorhabditis (3,000), Arabidopsis (500), Saccharomyces (900). Bacterial genomes comprise gene numbers which vary from approximately 500 to more than 8000. The number of essential genes in such genomes is unknown but can be estimated as being between 100 to 150 in smaller genomes, such as that of Haemophilus influenzae (1.83 Mb), to more than 500 in larger bacterial genomes, such as that of Pseudomonas aeruginosa (5.9 Mb). The potential and ramifications of using these essential genes and their products as novel therapeutic targets is enormous for the pharmaceutical industry and could open a new era in antimicrobial research. In addition, the identification of essential genes in higher life forms could provide important fundamental and practical information relating to cellular homeostasis, cancer and the like.

Powerful genetic techniques such as allelic replacement and gene knockouts have been developed. These technologies are effective but can only be applied to selected and candidate genes of interest. Applying these genetic techniques to whole genomes, even in the context of bacterial genomics, represents a highly inefficient and costly task and novel whole-genome based techniques and gene-screening assays must therefore be developed.

Comprehensive, rapid and simple screening of bacterial genomes for essential genes has not been possible because of the inability to identify mutants having an attenuated or displaying a lack of significant growth, within pools of mutagenized bacteria. It is also impractical to separately assess the significance of essential versus non-essential genes from each of the several thousand mutants necessary to screen a bacterial genome. Although genome-wide functional analysis appears to offer the best approach for the identification of dispensable versus essential genes, no simple, rapid and efficient identification method therefor has been forthcoming. Genome-based analyses provide primarily a functional classification rather than a detailed understanding of each gene. This is a critical aspect, especially in microbial genomics in which one can identify therapeutic targets by identifying essential genes.

U.S. Pat. No. 5,612,180 and Smith et al., 1995 (Proc. Natl. Acad. Sci. USA 92:6479-6483) teach a genetic footprinting method which, in essence, is a functional screen of genes under different selective conditions. A PCR-based method which enables functional analysis of genes is taught. Briefly, insertional mutagenesis is carried out on the genome to be tested. A portion of the DNA is subjected to a functional selection and a second portion subjected to non-selective conditions. The effect of the selection is then determined by amplifying the DNA isolated from the selected and non-selected populations. Differences in the presence or intensity of bands between the selection and non-selection conditions enable the functional analysis of a specific targeted gene or DNA region. The method which compares two populations of cells (selected vs non-selected) is based on the use of one set of primers for the PCR-based genetic footprinting: one primer binding to the insertional mutagen, the other being chosen arbitrarily as a unique sequence in the targeted region. This genetic footprinting method is unfortunately restricted to the identification of a correlation between a specific mutagenised region and of a specific phenotype. Furthermore, it lacks in providing a positive control of amplification originating solely from the targeted region (not from the insertional mutagen). Moreover, it is dependant on the discrimination of small differences in the extension products. Finally, it is based on the comparison of amplification products originating from two different sub-populations (selected vs non-selected).

There therefore remains a need to provide a simple and efficient method of identifying essential genes in a genome under non-selective conditions. There also remains a need to provide a simple and efficient method of identifying genes which are essential under specific conditions, the method providing an amplified signal originating solely from the non-mutagenised targeted region and in which amplification products from a single sub-population of cells are analysed. The present invention seeks to meet these and other needs.

### SUMMARY OF THE INVENTION

Accordingly, the present invention seeks to provide an essential gene test (EGT), an efficient and economical approach to define the function of thousands of sequences containing a complete open reading frame (ORF) or parts thereof, or known and/or unknown genes encoding hypothetical proteins or products. The EGT test is particularly effective at defining which sequences in databases contain an essential or a non-essential (dispensable) gene. In one embodiment the EGT assay is based on the premise that a mutation inactivating an essential gene should give rise in vivo, to a lethal phenotype irrespective of the growth conditions.

The present invention also seeks to provide an EGT test which enables the categorization of gene sequences as encoding essential and dispensable genes under selective conditions, the categorization being based on the analysis of a single sub-population of cells (“one tube population”).

Furthermore, the present invention seeks to provide an EGT test based on the detection of two basic types of extension products originating from two primer pairs.

By enabling an identification of essential genes in an organism, the EGT assays permits the identification of therapeutic targets in this organism. The present invention more preferably seeks to provide therapeutic targets in haploid organisms or haploid cells, particularly bacteria. In a particularly preferred embodiment, the invention seeks to provide therapeutic targets in bacterial strains in which insertional mutagenesis using mobile genetic elements is possible.

In accordance with one aspect of the present invention, there is provided a method for identifying essential and non-essential genes in a genome of a cell grown in non-selective conditions. This method comprises:

saturation mutagenesis of the genome by insertion mutagenesis, whereby an oligonucleotide sequence Is inserted in the target regions of the genome such that a population of cells having at least 90% of the target regions insertionally mutated is obtained;

growing the population of cells under non-selective conditions to provide a non-selected sub-population of cells;

amplifying a target region from the non-selected sub-population of cells, using a first primer which hybridizes to a known first end of the target region, and a second primer which hybridizes to another known end of the target region, the first and second primers thereby constituting a first primer pair, giving rise to a first extension product, and a third primer which hybridizes to the oligonucleotide sequence, the third primer constituting a second primer pair with the first or second primer, the second primer pair enabling the amplification of a second extension product; and

assessing for the presence or absence of the first and second extension product, whereby the presence of the first and second extension products is indicative of a non-essential gene, whereas the presence of the first extension product and the absence of the second extension product is indicative of an essential gene.

There is also provided a method for functional analysis of a target region in a sequence of interest. The method comprises:

mutagenizing the target region by insertion of a sequence tag to provide a population of DNA molecules containing a sequence tag insertion in at least 90% of nucleotide positions in the target region;

introducing the population of mutagenized DNA molecules into host cells that express the sequence of interest;

subjecting a first aliquot of the host cells to at least one selective condition and a second aliquot to a non-selective condition to provide at least one selected and one non-selected aliquot;

amplifying the target region from at least one selected and one non-selected aliquots, using a first primer hybridizing to the sequence tag and a second primer hybridizing to a known endpoint, the endpoint being characterized as an arbitrary unique sequence in the target DNA, to provide amplified DNA; and

resolving by gel electrophoresis the amplified DNA from at least one selected and one non-selected aliquots into individual bands differing by size to identify the position of individual sequence tag insertions within the target region,

whereby differences between the presence or intensity of bands between at least one selected and one non-selected aliquots are indicative that the sequence tag insertion causes a difference in response to the selective condition employed with at least one selected aliquot resulting in the functional analysis of the target region.

There is also provided a method for identifying essential and non-essential genes in a genome of a cell grown in non-selective conditions. This method comprises:

saturation mutagenesis of the genome by insertion mutagenesis, whereby an oligonucleotide sequence is inserted in the target regions of the genome such that a population of cells having at least 90% of the target regions insertionally mutated is obtained;

growing the population of cells under non-selective conditions to provide a non-selected sub-population of cells;

amplifying a target region from the non-selected sub-population of cells, using a first primer which hybridizes to a known end of the target region, and a second primer which hybridizes to the oligonucleotide sequence, the first and second primers constituting a primer pair capable of giving rise to an amplification of an extension product when the oligonucleotide sequence is inserted into the target region; and

assessing for the presence or absence of the first and second extension product, whereby the presence thereof is indicative of a non-essential gene, whereas the absence thereof is indicative of an essential gene.

In addition, there is provided a method for identifying essential and non-essential genes in a genome of a cell comprising:

saturation mutagenesis of the genome by insertion mutagenesis, whereby an oligonucleotide sequence is inserted in the target regions of the genome such that a population of cells having at least 90% of the target regions insertionally mutated is obtained;

growing the population of cells under selective or non-selective conditions to provide a selected or non-selected sub-population of cells;

amplifying a target region from the sub-population of cells, using a first primer which hybridizes to a known first end of the target region, and a second primer which hybridizes to another known end of the target region, the first and second primers thereby constituting a first primer pair, giving rise to a first extension product, and a third primer Which hybridizes to the oligonucleotide sequence, the third primer constituting a second primer pair with the first or second primer, the second primer pair enabling the amplification of a second extension product; and

assessing for the presence or absence of the first and second extension product, whereby the presence of the first and second extension products is indicative of a non-essential gene, whereas the presence of the first extension product and the absence of the second extension product is indicative of an essential gene.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

In the appended drawings:

FIG. 1 shows a summarized schematic representation of the essential gene test (EGT) according to the present invention by PCR using a single tube library of mutants. The primers are represented by arrows and genes essential (gene X) and non-essential (gene Y) by open-boxed lines indicated as ORF, the transposon miniTn5tet by the dark thick line. Dotted lines indicate transposon insertion into the gene to be tested. Abbreviations: PCR, polymerase chain reaction; F, Fx and Fy forward primers; Rx and Ry, reverse primers; ORF, open reading frame. The EGT is performed in an anchor primer method using a primer from the ORF either at the 5′ or 3′ end of the gene and the F primer from the transposon. Primer pairs Fx-Rx or Fy-Ry are used as controls to amplify the orfX or orfY;

FIG. 2 shows a schematic representation of the analysis of EGT products as generated from the primers and library of mutants as shown in FIG. 1. Products obtained by PCR are separated by agarose gel electrophoresis and transferred to a nylon membrane using the Southern method. Sensitivity is enhanced by hybridization using a DIG labelled probe of 398 bps from the tet gene of miniTn5tet.

FIG. 3 shows a physical and genetic map of the 2.2 Kb (kilobase) miniTn5tet element used. Numbers indicate nucleotide size (nts) delimited by vertical lines. Abbreviations: IR, inverted repeats; O , left IR of 19 nts; MCS, multiple cloning site; pHP45, DNA fragment from plasmid pHP45; tet, tetracycline resistance gene from plasmid pBR322; I, right inverted repeat of 19 nts. The dark horizontal arrows oriented inwards of tet (I) represent PCR primers giving rise to the 398 bps PCR product used as probe; the outward arrows indicate one of the two potential primers used in EGT.

FIG. 4 shows the results of a Southern-type gel hybridization of EGT PCR products separated by agarose gel electrophoresis using the DIG labelled 398 bps tet probe. The EGT was performed with Pseudomonas aeruginosa strain PAO1293 wild-type DNA and with DNA from a P. aeruginosa PAO1293 miniTn5tet library. Lanes: 1, PAO wild-type, ftsZ; 2, PAO miniTn5tet, ftsZ; 3, PAO wild-type, ampC; 4, PAO miniTn5tet, ampC; 5 PAO1 wild-type, asd; 6, PAO, miniTn5tet, asd; 7, PAO wild-type, ddl; 8, PAO miniTn5tet, ddl; 9, PAO wild-type, ftsA; 10, PAO miniTn5tet, ftsA; 11, PAO wild-type, ftsQ; 12, PAO miniTn5tet, ftsQ; 13, PAO wild-type, algK; 14, PAO miniTn5tet, aigk; 15, PAO wild-type rcf, 16, PAO miniTn5tet, rcf. Abbreviations: ftsZ, cell division protein, septation; ftsA, cell division; ampC, chromosomal beta-lactamase; asd, cell wall biosynthesis, aspartate semialdehyde dehydrogenase; ddl, cell wall biosynthesis, D-alanine ligase; ftsQ, cell division, algK, alginate biosynthesis; rcf, O-antigen polymerase.

Other objects, advantages and features of the present invention will become more apparent upon reading of the following non-restrictive description of preferred embodiments with reference to the accompanying drawing which is exemplary and should not be interpreted as limiting the scope of the present invention.

### DETAILED DESCRIPTION

The present invention relates to an essential gene test (EGT), which enables the identification of essential and dispensable genes in a genome under non-selective or selective conditions.

In one particular embodiment, the present invention provides the identification of essential and non-essential genes in a chosen genome using at least three oligonucleotide primers, constituting at least two primer pairs giving rise to a control extension product (generated from the non-mutagenized target region) and to an “experimental” extension product (generated from the mutated target region). In a preferred embodiment, the genome is a haploid genome and more particularly a bacterial haploid genome.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

The present description refers to a number of routinely used recombinant DNA (rDNA) technology terms. Nevertheless, definitions of selected examples of such rDNA terms are provided for clarity and consistency.

As used herein, “isolated nucleic acid molecule”, refers to a polymer of nucleotides. Non-limiting examples thereof include DNA and RNA molecules purified from their natural environment.

The term “recombinant DNA” as known in the art refers to a DNA molecule resulting from the joining of DNA segments. This is often referred to as genetic engineering.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” or “primer pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (i.e. DNA or RNA) for practising the present invention may be obtained according to well known methods.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 nucleotides, and they may be adapted to be especially suited to a chosen nucleic acid amplification system. As commonly known in the art, the oligonucleotide probes and primers can be designed by taking into consideration the melting point of hybridization thereof with its targeted sequence (see below, and in Sambrook et al., 1989, Molecular Cloning-A Laboratory Manual, 2nd Edition, CSH Laboratories; Ausubel et al., 1989, in Current Protocols in Molecular Biology, John Wiley & Sons inc., N.Y.). These two laboratory manuals are also examples of references teaching conventional methods, reagents, vectors, strains and the like (i.e. electrophoresis methods, blotting, sequencing, subcloning and the like) which can be used in the context of the present invention. Conventional methods in bacterial genetics are commonly known in the art. A non-limiting example of a reference teaching general techniques in bacterial molecular biology and basic manipulation of bacterial genetics include Miller 1972 (in Experiments in Molecular Genetics, Cold Spring Harbor Laboratory, CSH, NY).

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989 supra) and are commonly known in the art. In the case of a hybridization to a nitrocellulose filter, as for example in the well known Southern blotting procedure, a nitrocellulose filter can be incubated overnight at 65° C. with a labelled probe in a solution containing 50% formamide, high salt (5×SSC or 5×SSPE), 5× Denhardt's solution, 1% SDS, and 100 μg/ml denatured carried DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. High stringency conditions will be preferably used (Sambrook et al., 1989, supra).

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988 (Ann. Reports Med. Chem. 23:295) and Moran et al., 1987 (Nucl. Acids Res., 14:5019). Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Although less preferred, labelled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label is shown hereinbelow to be beneficial, by significantly increasing the sensitivity of the detection.

Furthermore, it enables automation. Probes can be labelled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe. In a particular embodiment, the EGT products were hybridized with a miniTn5tet hybridization probe labelled by the digoxigenin (DIG) method (i.e. in accordance with the Boehringer Mannheim's specifications).

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol l of E. coli in the presence of radioactive dNTP (i.e. uniformly labelled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately by the particular use thereof, and adapted accordingly by the person of ordinary skill. An oligonucleotide can be synthetised chemically or derived by cloning according to well known methods.

As used herein, a “primer” defines an oligonucleotide which is capable of annealing to a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, (Am. Biotechnol. Lab. 8:14-25). Numerous amplification techniques have been described and can be readily adapted to suit the particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188. In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analysed to assess whether the sequence or sequences to be detected are present. Detection of the amplified sequence may be carried out by visualization following EtBr staining of the DNA following gel electrophoresis, or using a detectable label in accordance with known techniques, and the like. For a review on PCR techniques (see PCR Protocols, A Guide to Methods and Amplifications, Michael et al., Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill.

Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696.

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise the a specific polypeptide or protein. It will be readily recognized by the person of ordinary skill, that the nucleic acid sequences of the present invention can be incorporated into anyone of numerous established kit formats which are well known in the art.

For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample (DNA or cells), a container which contains the primers used in the assay, containers which contain enzymes, containers which contain wash reagents, and containers which contain the reagents used to detect the extension products.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle, as described above, but designed to enable the expression of an inserted sequence following transformation into a host. The cloned gene (inserted sequence) is usually placed under the control of control element sequences such as promoter sequences. The placing of a cloned gene under such control sequences is often referred to as being “operably linked” to control elements or sequences.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

As used herein, the designation “functional derivative” denotes, in the context of a functional derivative of a sequence, whether nucleic acid or amino acid sequence, a molecule that retains a biological activity (either functional or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid has chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

Thus, the term “variant” refers herein to a protein or nucleic acid molecule which is substantially similar in structure and biological activity to the protein or nucleic acid of the present invention.

The functional derivatives of the present invention can be synthesized chemically or produced through recombinant DNA technology. All these methods are well known in the art.

As used herein, “chemical derivatives” is meant to cover additional chemical moieties not normally part of the subject matter of the invention. Such moieties could affect the physico-chemical characteristic of the derivative (i.e. solubility, absorption, half life and the like, decrease of toxicity). Such moieties are exemplified in Remington's Pharmaceutical Sciences (1980). Methods of coupling these chemical-physical moieties to a polypeptide are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutations of nucleic acid molecule is a mutant nucleic acid molecule. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in all other cellular components.

The mutagenesis of the DNA or of the cells is carried out in accordance with well-known methods (Sambrook et al., 1989, supra), such that the total DNA population or cell population has statistically at least one insertion mutation in each and every gene of the genome. Essentially, the one tube collection of mutants obtained by mutagenesis covers the complete genome. A typical mutagenesis experiment can yield mutants at frequencies varying from 10,000 clones to more than 1,000,000 clones. Such mutants can be recovered in a single tube. This mutagenesis scheme is based on the premise that the genome size is known, that mutagenesis is a random event and that a typical gene has an average size of 1 kilobase. For example and on a statistical basis, the 5.9 Mb Pseudomonas aeruginosa genome would require a minimum of 5,900 mutants to cover the genome at least once. This is herein defined as a 1×genome coverage. Thus, a collection of 17,500 mutants (3×), 29,500 mutants (5×) or 59,000 mutants (10×) could be utilized for screening in a typical EGT assay for this particular microorganism. Of course, and as shown in Example 2, the person of ordinary skill could also screen more than 10×. The person of ordinary skill will be able to adapt the present teachings to suit particular needs and adapt the instant invention to chosen genomes and specifics thereof.

A number of methods known to the person of ordinary skill can be used to mutagenize the genome of the chosen organism or population. Non-limiting examples include transposon-induced mutagenesis (as exemplified hereinbelow), the linker mutagenesis method as well as the restriction enzyme mediated integration method (REMI) (see for example in Directed Mutagenesis:

A Practical Approach, 1991, Edited by M. J. McPherson, 257 pps, The Practical Approach Series, IRL Press, Oxford University Press). Other non-limiting examples include oligonucleotide-directed mutagenesis, site-directed in vitro mutagenesis using uracil-containing DNA and phagemid vectors, phosphorothiotate-based, gapped-duplex, linker scanning and PCR based mutagenesis schemes using recombination. All these methods are well-known in the art.

In addition, a variation of the transposition process can be used such as, for example, the Primer Transposition kit (Perkin Elmer) based upon Tyl, the retrotransposon of Saccharomyces cerevisiae, but using the modified transposon supplied with the kit and designated as an artificial element At-2.

Thus, libraries can be constructed by a variety of methods and used in accordance with the EGT assay, provided that an insertional element enables the formation of a target sequence enabling genome amplification.

As used herein, the designation “therapeutic target” refers to any gene or product thereof that when blocked by known or novel molecules will affect the growth of the organism coding for the target.

As used herein, the designation “Non-selective conditions” refers to in vitro and/or in vivo growth conditions wherein all the parameters and factors which are required for optimal growth are present. Non-limiting examples of such parameters/factors include growth media nutrients, temperature, pH, cell line, and the like. Under such conditions, one would expect the organism to be maintained prior to the mutagenesis step.

As used herein, the designation “Selective conditions” refers to conditions which are defined by the nature of the experiment done in vitro and/or in vivo and in which one specific parameter or factor or set of conditions are modified (in comparison to non-selective conditions) to determine if essentials genes or gene products can be identified in that particular condition. A non-limiting example of a selective condition includes growth at a restrictive temperature (i.e. temperature sensitive or ts).

It will be clear to the person of ordinary skill, that insertional mutagenesis of an essential gene, within the context of a cell, will result in the death of that cell. Consequently, the genome of this particular cell will not be available as a substrate for the amplification process in accordance with the EGT method of the present invention.

The DNA molecule analysed may be a gene, a fragment thereof cloned into a vector or preferably a genome.

It will also be understood that the instant invention is not limited to the identification of essential ORFs. A person of ordinary skill will understand that insertions into 5′ and 3′ non-coding sequences could also be shown to be detrimental or fatal to the survival of a cell harboring such an insertion. Thus, the present invention also covers the identification of DNA targets which are essential under selective or non-selective conditions.

As used herein, the terminology “target region” defines a DNA region for which preliminary sequence data is available sufficiently to enable the design of a first primer pair which will, under appropriate conditions, give rise to a recognizable extension product. The target region is determined and defined by the available sequence data available for the particular genome analysed, and by the limits in the amplification method used. For PCR, for example, the conditions permit extension products to reach about 2000 nucleotides. The target region should thus be between about 50 to about 2000 nucleotides. Preferably between about 200 and about 1000. Since sequence information can be clustered, some genes might have several target regions. In any event, the mutagenesis conditions should be adapted so as to enable an insertional mutagenesis of all targeted regions. In essence, a person of ordinary skill will adapt the mutagenesis scheme so as to permit saturation mutagenesis of the DNA to be analysed.

Although in a preferred embodiment, the present invention is adapted for use with a whole genome, a DNA molecule inserted into a vector can also be used in accordance with the present invention. In such an embodiment, the vector should permit an expression of the DNA molecule in order to permit an assessment of the essentiality of the gene product. In such a scheme, it will be understood that only dominant insertional mutation can provoke the lethality since, presumably, a copy of a wild type or homologous copy of the gene which is present on the vector, is present in the host cell. Consequently, it will be clear to the person of ordinary skill that although the present invention is not limited to haploid genomes, the method of the present invention is favorably used in a context of a haploid organism, more preferably a haploid microorganism and especially in Gram positive and Gram negative bacteria. Organisms in which conversion to homozygocity is efficient and/or complete are also covered by the scope of the present invention. In a preferred embodiment therefore, prokaryotic genomes and lower eukaryotic genomes such as the haploid genomes of parasites and protista are used. Non-limiting examples of such lower eukaryotic genomes include that of tachyzoite form of Toxoplasma gondii, of Plasmodia, Schistosoma and Leishmania species, as well as those of fungi such as that of Candida, Aspergillus, Neospora and other disease causing (in plants, in animals and in humans) relevant fungi are especially preferred genomes. In addition, all disease causing agents such as Influenzae, HIV, Herpes and other viruses may also be used in the context of the present invention.

It will also be understood by the person of ordinary skill that the methods of the present invention can also be adapted to identify essential and dispensable genes or target regions in eukaryotes such as mammalian or plant cells. In a preferred embodiment, haploid mammalian or plant cells will be used in accordance with the present invention. In an especially preferred embodiment, the haploid cells are gametes.

It shall be understood that although the saturation insertional mutagenesis of the present invention is carried out by a shotgun approach (without specifically directing the insertion to specific sequences), a rational design of insertion mutation could also be carried out, especially with DNA molecules inserted into vectors.

Since the design of some of the primers (i.e. the first primer pair) depends on known sequence data from the genome to be analysed, it follows that minimum stretches of sequence data must be available in order to enable the EGT method of the present invention. Preferably, contiguous nucleic acid sequence data of approximately twelve nucleotides, to approximately twenty-four nucleotides in the targeted region must be available.

Although in a preferred embodiment, the method of the present invention relates particularly to genomes of organisms which do not contain or contain few introns, the present invention could be adapted by a person of ordinary skill for intron-containing genomes. Briefly, the level of mutagenesis would have to be increased in order to enable saturation to occur. Saccharomyces cerevisiae is one non-limiting example of an organism which contains introns.

The terminology “genomic profiling” is used herein to include an amplification of one or more genes (an operon in bacteria, for example). The length of the target region to be amplified will have to be considered in adapting the conditions of the amplification methods, as commonly known.

Numerous insertional mutagenesis method are known in the art. It will be clear to the person of ordinary skill that the method should be adapted to enable the insertion of the sequence which is complementary to that of a primer binding thereto (described herein in some instances as primer number 3).

The term “saturation mutagenesis” as used herein with reference to a genome, refers to an insertion mutagenesis in substantially every gene thereof and/or every target region thereof. Based upon statistical analysis and well known methods, at least 90%, preferably, 95% and more preferably 100% of the genes and/or target regions will have been mutagenised. Briefly, to estimate the required conditions enabling the aiming of a complete population of mutagenised genes, the statistical analysis utilised is based on a number of criterions: 1) a completely random insertion of the insertion element (i.e. a mobile element); 2) an average size of 1 Kb for a typical gene in a prokaryote genome; 3) knowledge a priori of the genome size (Megabases). For example, a complete 1×coverage of the P. aeruginosa 5.9 Mb genome would require a minimum of about 6000 clones after the mutagenesis experiment. Preferably, a minimum of 10×coverage of the genome should be used by using 60,000 clones. When relating to DNA molecules present on a vector, saturation mutagenesis refers preferably to the insertion element being present at every nucleotide position thereof. It will be clear to a person of ordinary skill to which the instant invention pertains, that the estimation of the conditions can be readily adapted to meet variations in the above-mentioned criterions or to meet particular needs should the criterions be different.

Mutational methods include, without being limited thereto, insertional mutations in which a DNA molecule is inserted without loss of native sequences, or substitutional mutations in which the DNA molecule inserted replaces native DNA molecule of the targeted region.

It shall be understood that the choice of a particular insertional element can be adapted to particular needs, provided that it is absent from the genome which is to be analysed, that it is sufficiently long to permit the generation of a primer which binds thereto (hence the need for known sequence data of about 12 contiguous nucleotides for the primer target on the genome), and disrupts the gene or target region it is inserted into. In a preferred embodiment, the insertional mutagenesis is provided by an insertional element such as a transposon or genetic mobile element (i.e. Tn5, miniTn5tet, Tn10, Tn916, Tn917, Ty, the AC and OS maize elements, Ecopia, the P element and derivatives of these mobile elements). In such cases, the insertional mutagenesis will be carried out with the insertional elements in accordance with known methods.

Insertional mutagenesis of DNA can also be carried out by using the integrases protein of retroviruses to mediate the insertion of a selected primer into a target region. Following amplification, the amplified product or extension product can be detected. In a preferred embodiment, such products can be sized-fractionated by gel electrophoresis as well known in the art. In another embodiments the extension products can be detected after separation on columns and the like. Hybridization, capture and the triplex DNA technology are non-limiting examples of technologies which could be used to detect the amplified products (Lanbiewicz et al., 1997, Nucl. Acids Res. 25; 2037-38; and Ito et al., 1992, Proc. Natl, Acad. Sci 89: 495-8).

In a particular embodiment, the amplification is carried out by the PCR method using an anchor primer method (Dieffenback et al., 1995 in PRC Primer, A Laboratory Manual, Cold Spring Harbor, CSH Press, NY).

In a particular embodiment, a kit for identifying essential genes in a genome contains at least three oligonucleotide primers, constituting at least two primer pairs, a mutated genome, and solutions for enabling hybridization between the mutated genome sequences and the oligonucleotide primers and for enabling amplification of the extension product. Oligonucleotide primers can be suspended in solution or provided separately in lyophilized form. The components of the kit can be packaged together in a common container. The kit typically includes an instruction sheet for carrying out a specific embodiment of the method of the present invention. Additional optional components of the kit include detection probes, and means for carrying out a detection step (for example, a probe or primer is labelled with a detectable marker).

Insertional Mutagenesis of the Targeted Genome

First, insertional mutagenesis must be performed so as to cover most if not all genes of a particular genome in a population of cells. Under these conditions, one would expect the one tube mutagenized population to cover the spectrum of each and every gene coded by a particular organism.

Insertional Mutagen

In one embodiment in which a bacterial genome is targeted, a bacterial population is mutagenized using for example a mobile element having a high frequency of transposition (such as, for example, Tn5, miniTn5tet, Tn10, Tn916, Tn917, IS elements or any other known mobile genetic element) creating insertional mutations at diverse sites. Depending on the conditions and mobile element utilized, one may produce a single tube population containing cells having an insertion in essentially all the genes. Any particular type of mutagenesis scheme including insertion elements, PCR mutagenesis, random insertion of DNA by synthetic or biological methods would be amenable to genetic analysis by the EGT test or assay.

The assay can also be applied to any simple organisms such as viruses. The EGT finds utility in disease causing viruses from plants, from animals and from humans. Non-limiting examples include the potato blight virus in plants, the equine encephalitis virus in animals and the cytomegalovirus in humans. Additional examples include single eukaryotic cells of fungi and of yeasts causing diseases such as mycoses and include for example Candida, Cryptococcus, Histoplasma, Blastomyces, Coccioides, Aspergillus, Fusarium, and Trychophyton, and the like. Thus, the EGT assay could be applied to all disease causing organisms (See the listing of the Manual of Clinical Microbiology, 1995, ASM Press). The person of ordinary skill will readily adapt the EGT accordingly. For the targeting of the yeast genome the insertional element Ty is a representative example of an insertional mutagen which can be used in accordance with the present invention. In addition, the EGT assay can be utilized to dissect metabolic and genetic pathways by assessing mutagenized populations in different in vitro and in vivo conditions.

Amplification

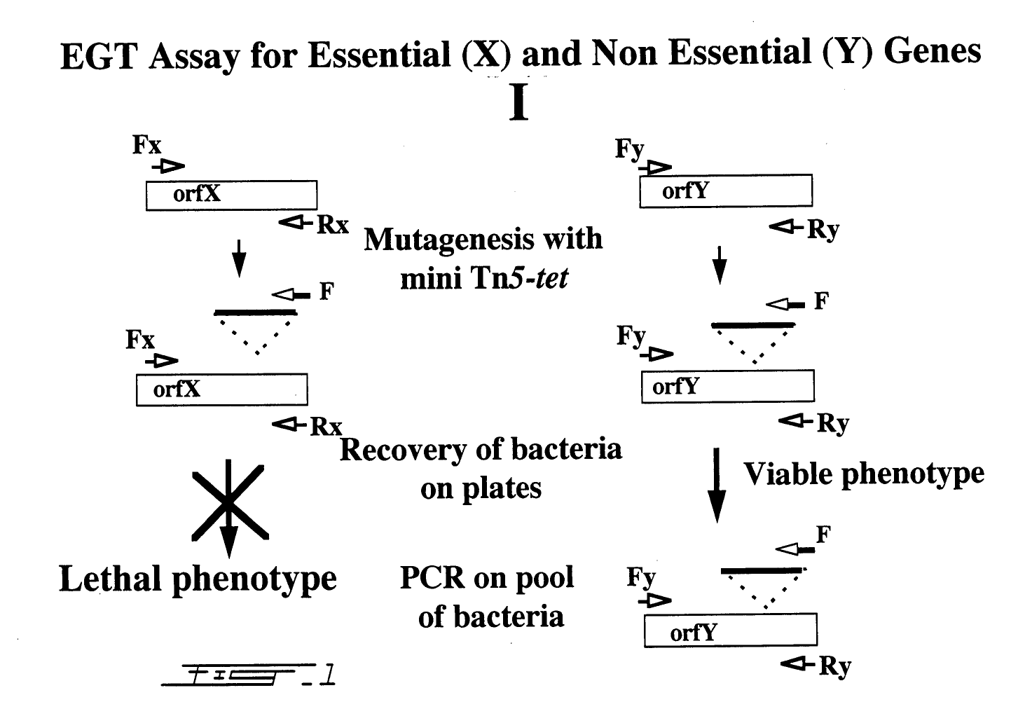

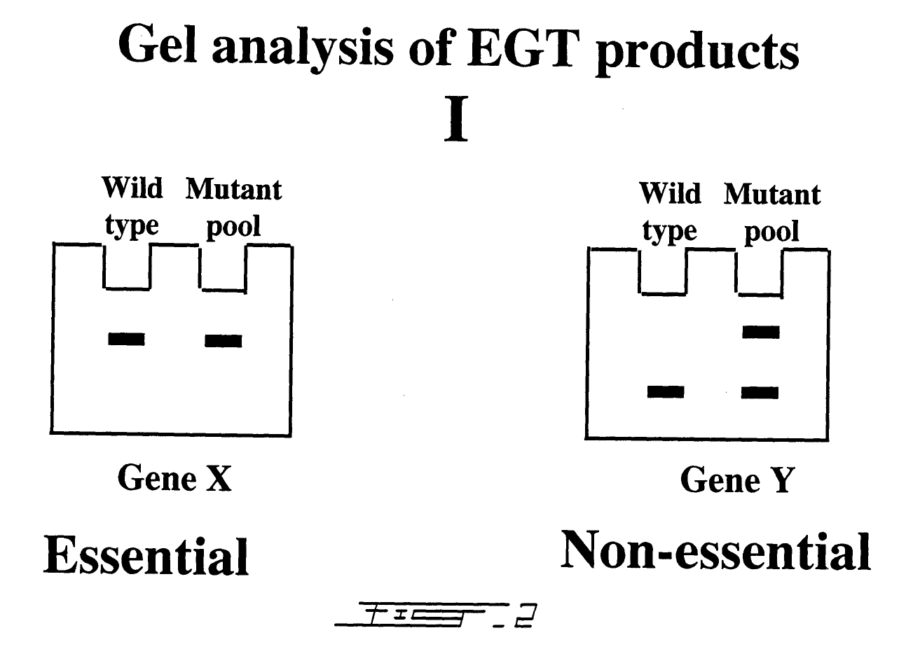

A sample of the mutagenized population is then submitted to nucleic acid amplification. In a preferred embodiment, the amplification is carried out by PCR using either cells directly or by preparing an aliquot of DNA from the cells (such PCR methods are well known in the art). A collection of two primers specific for the sequence under investigation (from a genomic database and assumed to encode an essential or dispensable gene where only part of the ORF is known) and defining a first primer pair, gives rise to an amplification product of a defined size (or control extension product). A third primer specific for the insertional mutagen is also used. This three primer assay will give specific amplification products defining a sequence as essential or dispensable. The EGT assay was performed as summarized in FIG. 1 using a wild-type and a mutagenized population. The role of a particular sequence as essential or dispensable is visualized as the presence (non-essential) or depletion of defined satellite amplification products (essential) (FIG. 2). It shall be clear that the performance of EGT with the wild-type population is not necessary ‘per se’ since the target region of the insertional element should not be present in the population prior to its mutagenesis therewith.

Interpretation of the results of EGT assay

The primer pairs selected from the sequence of interest defines an amplification product that will be present both in essential genes and in dispensable genes irrespective of the growth conditions since in the context of a population of cells, individual cells having no insertions in the targeted sequence of interest will always be present. Thus, the first primer pair serves as an internal control for the assay conditions (FIGS. 1 and 2). If the insertion occurs in a dispensable gene, the second primer pair, constituted by a primer specific for the targeted sequence and a primer specific for the insertional mutagen, gives rise to a specific extension product and a series of additional band products. Thus, in addition to the expected product originating from the first primer pair (or control extension product), additional amplification products will be visible (FIG. 2). The difference in the size of the additional product will reflect the distance between the target region of the third primer (the insertion “point”) and that of the first primer (or second primer). In contrast, insertion of an element in an essential gene will not yield an amplification product (lethal phenotype) and the only visualized amplification product will be generated by the amplification of mutagenized cells containing no insertions in the essential sequence of interest (originating from the first primer pair) (FIG. 2).

As alluded to above, the EGT assay enables automation. For example, by using fluorescent primers (labelled with distinct fluorochromes) the EGT assay could be used in conjunction with the ABI GENESCAN.

The following examples are offered by way of illustration and not by way of limitation.

### EXAMPLE 1

EGT Assay Using Two Primer Pairs on Two Pseudomonas Aeruginosa Genes The EGT assay was applied to the Pseudomonas aeruginosa strain PAO1 5.9 Mb genome in the following way. First, a library of insertion mutants was constructed with the miniTn5 Km insertion element using standard methods. A collection of 60,000 clones (10×genome coverage) obtained were pooled into a single tube.

A first primer pair of 21-mers specific and internal to the ftsZ gene sequence (ftsZ:5′-ATC ACC ATC CCG MC GAG MG-3′ SEQ ID NO:1) and (ftsZ2:5′-TAT CCA GGT MT CCA GGT CAT-3′ SEQ ID NO:2) give a 669 bps amplified PCR product. The PCR conditions for DNA amplification were carried out in accordance with the manufacturer's recommendations (Perkin Elmer Cetus and Applied Biosystems) using a DNA sample preparation. In a typical EGT assay, one would expect the 669 bps to be present irrespective of the mutagenesis or growth conditions.

The EGT assay was performed for ftsZ by using the following primers: (KanaputR1: 5′-GCG GCC TCG AGC MG ACG TTT-3′ SEQ ID NO:3) and (KanaputF4: 5′-TTG GTT GTA ACA CTG GCA GAG-3′ SEQ ID NO:4) in combination with one and\or the two above-mentioned primers (ftsZ1 and ftsZ2). The result of the EGT assay showed a product of 669 bps and no satellite bands, irrespective of the mutagenesis scheme. Thus, only the first primer pair gave rise to an extension product. Thus, ftsZ is therefore defined as an essential gene by the EGT method.

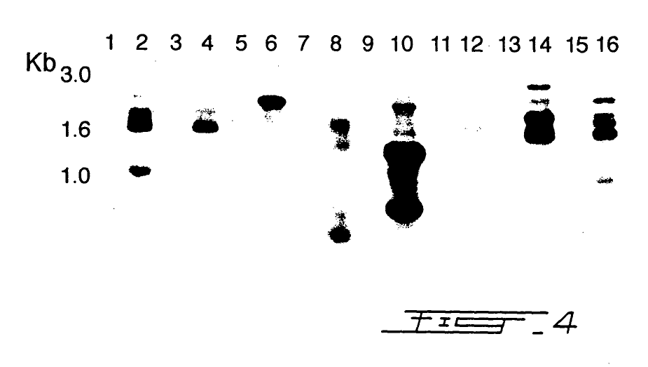

The EGT assay was tested with the ampC gene using primers (ampcF1: 5′-CAT CGC TTC CAC ACT GCT-3′ SEQ ID NO:5) and (ampcR1: 5′-TGC CGG GM CAC TTG CTG CTC-3′ SEQ ID NO:6) constituting a first primer pair giving rise to a PCR product of 592 bps irrespective of the mutagenesis. When used in conjunction with the KanaputR1 and KanaputF1 primers, a PCR product of 592 bps (positive control) and additional DNA bands (due to insertions in the ampC gene) could be visualized in the agarose ethidium bromide stained gel. Thus, the EGT assay would define the ampC gene as non-essential. However, repeated experiments showed that the absence of an extension product from ftsZ and miniTn5 Km was most likely explainable by the fact that a P. aeruginosa strain had become kanamycin resistant while not containing miniTn5 km. Such artifactual km resistances have been previously described (Cornelio et al., 1992, J. Gen. Microbiol. 138:1337-1343). It has also been found that P. aeruginosa contains more than one copy of ftsZ (see FIG. 4; lane 2, showing bands at 1.75 and 2.0 kb).

In any event, Example 1 still demonstrates the principle of the EGT assay using at least 2 primer pairs. In order to clearly demonstrate the potential of EGT for identifying an essential gene and hence a potential therapeutic target, another insertion element (which does not have a propensity to give false positive results) was used.

### EXAMPLE 2

Validation of the EGT Assay Using Pseudomonas Aeruginosa Genes

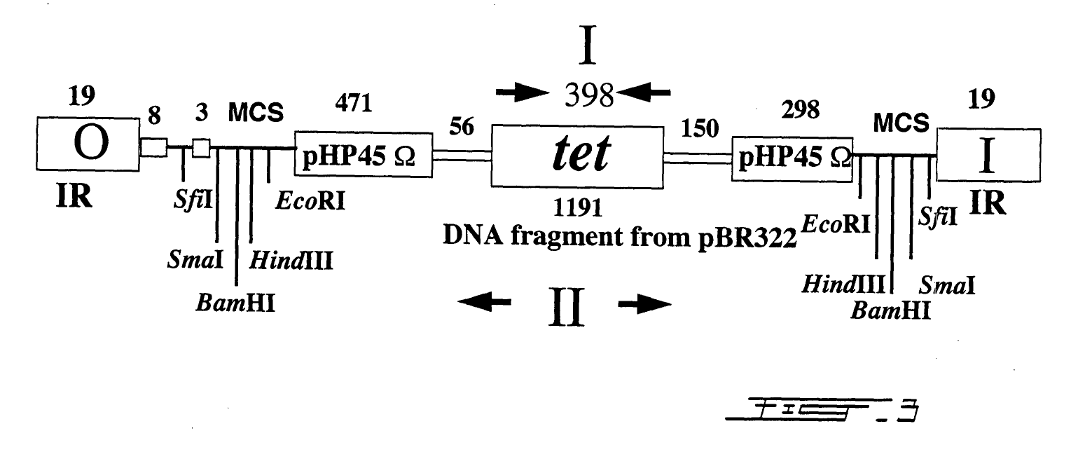

The EGT assay was used with the Pseudomonas aeruginosa strain PAO1293, a chloramphenicol resistant derivative of the completely sequenced PAO1 (5.9 Mb). Strain PAO1293 was mutagenized with the 2.2 Kb miniTn5tet transposable element (FIG. 3). Briefly, an E. coli donor strain (tra+, RP4, Mob+) was used to transfer the putminitn5tet into P. aeruginosa strain PAO1293 by conjugation in accordance to well-known methods. Several libraries were obtained in optimized conditions. For example, a conjugation method was used to transfer the miniTn5 into P. aeruginosa but in condition where the transfer is at a high frequency of DNA transfer. Growth of the P. aeruginosa recipient was carried out at 43° C. to eliminate the restriction modification system and facilitate transfer. A ratio of 1:10 donor recipient cells with matings was used and performed on solid agar (rich or defined media).

The complexity of mutant libraries was assessed by comparing of clone frequencies (tet resistant cells per known concentration of recipient cells) when obtained plated on rich and synthetic complete or minimal media. The number of tet resistant clones represent an estimation of the frequency of mutants obtained. One library retained for EGT analysis contained 92,260 Tetracycline resistant clones. The library was characterized using four criterias including: 1) estimation of the frequency of ex-conjugant mutants (cells having received the miniTn5tet and being tet resistant); 2) genomic profiling of 30 Tetracycline resistant (tetR) clones selected via PCR amplification of an internal 398 bps tet gene product; 3) Southern-type gel hybridization using the 398 bps tet fragment as probe. Briefly, the 398 bps PCR product shown between the arrows in FIG. 3 was labeled with DIG. Hybridization was carried out on 30 tet resistant clones randomly selected from the library. The Southern hybridization data showed a single hybridization signal in the genomic DNA of each clone and of a distinct size in each case (data not shown). The library was also tested using PCR with the primers represented by the arrows in FIG. 3 and giving the 398 bps product. Again, 30 clones were randomly chosen and PCR amplification yielded a positive PCR result in 28 of the 30 clones (data not shown); and 4) sequencing of the Tn5tet insertion endpoints for 30 clones. Based on these criterias and biostatistical analysis as a binomial probability (Binomial distribution, pps. 82-104, in: Fundamentals of Biostatistics, 1995 by Bernard Rosner, 4th Edition, Duxbury Press, An Imprint of Wadsworth Publishing Company, Boston, USA), binomial in the sense that the presence of miniTn5tet is estimated as a yes or no, the library was estimated to cover the 5.9 Mb chromosome at 15.6×genome equivalents. Thus, EGT screening should identify a gene as essential or dispensable at a frequency between 85% up to 100%. Indeed, if one extrapolates that 28/30 clones is the lowest probability for 92,260, this gives 84%; if 30/30 is used as the highest value and 15.6×genome equivalent, then it is 100%.

From the DNA sequence data available for P. aeruginosa representing the complete genome with 85,000 sequencing reactions, from gene sequences done in the laboratory and available in the literature (i.e. the Pathogenesis Corporation's Website), PCR primers were designed from the 5′ and 3′ ends (but coding sequences) for each of the gene selected.

A collection of 8 genes (including ddl implicated in D-alanine ligase, rcb affecting LPS; and algK involved in alginate biosynthesis) were selected as positive/negative controls. The primers listed below in Table 1 were selected so as to give a PCR product which would cover the most part of an open reading frame (ORF) or gene. With such primers, it is expected that EGT will yield an amplified product having a detectable change in size, whether it initiates from a dispensable gene or whether it originates from the gene having no insert. The general principles that guided the design of the primers are well-known in the art. Briefly, the primers were selected from the known sequence of each gene and as a PCR primer pair preferably capable of amplifying the complete ORF (from the initiation to the termination codon), taking into consideration that primers would not give rise to secondary structures and having melting temperatures (Tm) that would not differ for more than 2 degrees for a given gene to be tested by EGT. This was done with the OLIGO (version 4.03) Primer Analysis Software, Wojciech Rychlik, National Biosciences Inc., Plymouth, Mn. USA. The sequence of two 21-mer primers derived from the miniTn5tet element is also shown in Table 1.

TABLE 1

| | |
|---|---|
| 1.ftsZ gene: | |
| Primer 1: ftsZ3 | |
| 5′CAT CGC ACA AAC CGC CGT CAT 3′ | SEQ ID NO:7 |
| Primer 2: ftsZ4 | |
| 5′ACG CAG GAA CGC CGG GAT ATC 3′ | SEQ ID NO:8 |
| 2. ampC gene: | |
| AmpCF2 | |
| 5′CAT CGC CGG TTC CAC ACT GCT 3′ | SEQ ID NO:9 |
| AmpCR2 | |
| 5′GCT GAG GAT GGC GTA GGC GAT 3′ | SEQ ID NO:10 |
| 3. Asd gene: | |
| AsdF2 | |
| 5′TCA CCA CGT CGA ACG TCG GTG 3′ | SEQ ID NO:11 |
| AsdR2 | |
| 5′CTC CAG CAG GAT GCG CAA CAT 3′ | SEQ ID NO:12 |
| 4. ddl gene: | |
| ddl3 | |
| 5′AAG TCC GGC GCG ATG GTC CTG 3′ | SEQ ID NO:13 |
| ddl4 | |
| 5′GCC AGG ATC GCC AGC ACC AGT 3′ | SEQ ID NO:14 |
| 5. ftsA gene: | |
| ftsAF1 | |
| 5′GCA GAG CGG CAA GAT GAT CGT 3′ | SEQ ID NO:15 |
| ftsAR1 | |
| 5′CTT GGG TTC GTC GCT GCT GTA 3′ | SEQ ID NO:16 |
| 6. ftsQ gene: | |
| ftsQ3 | |
| 5′TGG CGT ACT GCT CCG TCA TCA 3′ | SEQ ID NO:17 |
| ftsQ4 | |
| 5′TTG GGG TAA CGC AGG TCG ATC 3′ | SEQ ID NO:18 |
| 7. gene aIgK (GenBank no.: X99206) | |
| algK1 | |
| 5′GCC ACC GCC CAG AGC AAC TAC 3′ | SEQ ID NO:19 |
| algK2 | |
| 5′CTG GCT CTG CAG CAG GCT GAG 3′ | SEQ ID NO:20 |

8. gene rcf serotype O2 (GenBank no.: U50599)

| | |
|---|---|
| rc1 | |
| 5′GCT CGA GTC GAC AGG TCT ATT 3′ | SEQ ID NO:21 |
| rcf2 | |
| 5′GCG CAA GGA AAA GCA GTA TCA 3′ | SEQ ID NO:22 |
| miniTn5tet | |
| TetF1 | |
| 5′CACCGTCACCCTGGATGCTGT 3′ | SEQ ID NO:23 |
| TetR1 | |
| 5′CCATACCCACGCCGAAACAAG 3′ | SEQ ID NO:24 |

One advantage of using primers which should yield an amplification product spanning virtually the whole gene, is that it decreases the probability of missing fatal or detrimental insertions.

The complexity of the EGT results was simplified by using a single primer pair from above, namely the first primer from each gene and TetF1 (one from the gene and one from the transposon and called insert anchored primers).

The results obtained are shown in FIG. 4 and are representative of 3 distinct experiments. The EGT was performed using these known genes. The DNA was amplified by PCR. Briefly, PCR reactions were done in 50 μl volume containing 1.5 mM MgCl2, 200 nM primers, 200 mM dNTPs, 20 mM Tris, pH 8.4, 50 mM KCl using 30 cycles of amplification in a Perkin Elmer Thermal cycler. The programmed cycles were 30 cycles of 1 min at 95° C., 1 min at 60°C., 2 min at 72° C., one elongation step of 7 min at 72° C. and a soak at 4° C. For example, genes such as asd should be considered essential because an insertional mutation would give a lethal phenotype; while others such as ampC, algK and rcf are well documented to be non-essential genes, i.e. mutants are readily available. The situation for ftsZ, ddl, ftsQ and ftsA is not clear but they are important genes implicated in cell division and in cell wall biosynthesis. As depicted in FIG. 4, lane 6, the EGT clearly identified the asd gene as essential; all other genes tested gave multiple bands representing insertions in different positions for each gene and would therefore be considered non-essential.

Thus, the EGT can be used to identify essential genes in the absence of selection conditions. For certainty, the EGT assay could also be adapted to identify essential genes under selective conditions.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The instant description refers to a number of documents, the content of which is herein incorporated by reference.

What is claimed is:

1. A method for identifying essential and non-essential genes in a genome of a cell grown in non-selective conditions, said method comprising:
saturation mutagenesis of said genome by insertion mutagenesis, whereby an oligonucleotide sequence is inserted in the target regions of said genome such that a population of cells having at least 90% of said target regions insertionally mutated is obtained;
growing said population of cells under non-selective conditions to provide a non-selected sub-population of cells;
amplifying a target region from said non-selected sub-population of cells, using a first primer which hybridizes to a known first end of said target region, and a second primer which hybridizes to another known end of said target region, said first and second primers thereby constituting a first primer pair, giving rise to a first extension product, and a third primer which hybridizes to said oligonucleotide sequence, said third primer constituting a second primer pair with one said first or second primer, said second primer pair enabling the amplification of a second extension product; and
assessing for the presence or absence of said first and second extension product, whereby the presence of the first and second extension products is indicative of a non-essential gene, whereas the presence of the first extension product and the absence of the second extension product is indicative of an essential gene.

2. A method according to claim 1, wherein mutagenizing is performed with a transposable element.

3. A method according to claim 2, wherein said target DNA comprises a gene encoding a protein.

4. A method for functional analysis of a target region in a sequence of interest, said method comprising:
mutagenizing said target region by insertion of a sequence tag to provide a population of DNA molecules containing a sequence tag insertion in at least 90% of nucleotide positions in said target region;
introducing said population of mutagenized DNA molecules into host cells that express said sequence of interest;
subjecting a first aliquot of said host cells to at least one selective condition and a second aliquot to a non-selective condition to provide at least one selected and one non-selected aliquot;
amplifying said target region from said at least one selected and one non-selected aliquots, using a first primer hybridizing to said sequence tag and a second primer hybridizing to a known endpoint, said endpoint being characterized as an arbitrary unique sequence in said target DNA, to provide amplified DNA; and
resolving by gel electrophoresis said amplified DNA from said at least one selected and one non-selected aliquots into individual bands differing by size to identify the position of individual sequence tag insertions within said target region,
whereby differences between the presence or intensity of bands between said at least one selected and one non-selected aliquots are indicative that said sequence tag insertion causes a difference in response to said selective condition employed with said at least one selected aliquot resulting in the functional analysis of said target region.

5. A method according to claim 4, wherein mutagenizing comprises the steps of:
combining DNA comprising said target region with retroviral integrase and a first set of complementary oligonucleotide primers, said primers comprising (a) a recognition sequence for said retroviral integrase and (b) a sequence tag, wherein said retroviral integrase mediates the insertion of said first set of complementary oligonucleotide primers to provide a population of mutagenized DNA molecules.

6. A method according to claim 4, wherein mutagenizing comprises the steps of:
combining DNA comprising said target region with retroviral integrase and a first set of complementary oligonucleotide primers, said primers comprising (a) a recognition sequence for said retroviral integrase and (b) a recognition site for a type IIs restriction endonuclease, wherein said retroviral integrase mediates the insertion of said first set of complementary oligonucleotide primers to provide a population of mutagenized DNA molecules
cutting said population of mutagenized DNA molecules with said type Ils restriction endonuclease to provide cut DNA; and
ligating to said cut DNA a second set of complementary oligonucleotide primers comprising a sequence tag.

7. A method according to claim 5, wherein said sequence of interest comprises a gene encoding a protein.

8. A method according to claim 4, wherein said selective condition is growth of cells in media lacking a nutrient that is an intermediate in a metabolic pathway.

9. A method according to claim 8, wherein said population of mutagenized DNA molecules are cloned into a filamentous bacteriophage vector with regulatory sequences for expression of said sequence of interest.

10. A method according to claim 5, wherein said sequence of interest comprises a regulatory gene.

11. A method according to claim 10, wherein said selective condition is growth in media containing a cytotoxic agent, and said regulatory gene controls expression of a gene conferring resistance to said cytotoxic agent.

12. A method according to one of claims 4 to 11, whereby the absence of a band under said selective condition and its presence under non-selective conditions is indicative of a target region which is essential under said selective condition.

13. A method according to one of claims 1-12, wherein said genome is a haploid genome.

14. A method according to claim 13, wherein said haploid genome is a bacterial genome.

15. A method for identifying essential genes in a genome of a cell grown in non-selective conditions, said method comprising:
saturation mutagenesis of said genome by insertion mutagenesis, whereby an oligonucleotide sequence is inserted in the target regions of said genome such that a population of cells having at least 90% of said target regions insertionally mutated is obtained;
growing said population of cells under non-selective conditions to provide a non-selected sub-population of cells;
amplifying a target region from said non-selected sub-population of cells, using a first primer which hybridizes to a known end of said target region, and a second primer which hybridizes to said oligonucleotide sequence, said first and second primers constituting a primer pair capable of giving rise to an amplification of an extension product when said oligonucleotide sequence is inserted into said target region; and
assessing for the presence or absence of said first and second extension product, whereby the presence thereof is indicative of a non-essential gene, whereas the absence thereof is indicative of an essential gene.

16. A method for identifying essential genes in a genome of a cell comprising:
saturation mutagenesis of said genome by insertion mutagenesis, whereby an oligonucleotide sequence is inserted in the target regions of said genome such that a population of cells having at least 90% of said target regions insertionally mutated is obtained;
growing said population of cells under selective or non-selective conditions to provide a selected or non-selected sub-population of cells;
amplifying a target region from said sub-population of cells, using a first primer which hybridizes to a known first end of said target region, and a second primer which hybridizes to another known end of said target region, said first and second primers thereby constituting a first primer pair, giving rise to a first extension product, and a third primer which hybridizes to said oligonucleotide sequence, said third primer constituting a second primer pair with one said first or second primer, said second primer pair enabling the amplification of a second extension product; and
assessing for the presence or absence of said first and second extension product, whereby the presence of the first and second extension products is indicative of a non-essential gene, whereas the presence of the first extension product and the absence of the second extension product is indicative of an essential gene.

17. A method according to claim 16, wherein said genome is a haploid genome.

18. A method according to claim 16 to 18, wherein insertion mutagenesis is carried out with a transposable element.

19. A method according to one of claims 1-18, wherein said amplification is carried out by the polymerase chain reaction.

20. A method for identifying a therapeutic target in a genome of a cell grown in non-selective conditions, said method comprising:
saturation mutagenesis of said genome by insertion mutagenesis, whereby an oligonucleotide sequence is inserted in the target regions of said genome such that a population of cells having at least 90% of said target regions insertionally mutated is obtained;
growing said population of cells under non-selective conditions to provide a non-selected sub-population of cells;
amplifying a target region from said non-selected sub-population of cells, using a first primer which hybridizes to a known first end of said target region, and a second primer which hybridizes to another known end of said target region, said first and second primers thereby constituting a first primer pair, giving rise to a first extension product, and a third primer which hybridizes to said oligonucleotide sequence, said third primer constituting a second primer pair with one said first or second primer, said second primer pair enabling the amplification of a second extension product; and
assessing for the presence or absence of said first and second extension product, whereby the presence of the first extension product and the absence of the second extension product is indicative of an essential gene and hence of an identification of a therapeutic target in said cell.


SEQUENCE LISTING (24 sequences)

SEQ ID NO: 1 - DNA, 21 bp, Artificial Sequence
  Description of Artificial Sequence synthetic oligonucleotide
atcaccatcccgaacgagaag

SEQ ID NO: 2 - DNA, 21 bp, Artificial Sequence
  Description of Artificial Sequence synthetic oligonucleotide
tatccaggtaatccaggtcat

SEQ ID NO: 3 - DNA, 21 bp, Artificial Sequence
  Description of Artificial Sequence synthetic oligonucleotide
gcggcctcgagcaagacgttt

SEQ ID NO: 4 - DNA, 21 bp, Artificial Sequence
  Description of Artificial Sequence synthetic oligonucleotide
ttggttgtaacactggcagag

SEQ ID NO: 5 - DNA, 18 bp, Artificial Sequence
  Description of Artificial Sequence synthetic oligonucleotide
catcgcttccacactgct

SEQ ID NO: 6 - DNA, 21 bp, Artificial Sequence
  Description of Artificial Sequence synthetic oligonucleotide
tgccgggaacacttgctgctc

SEQ ID NO: 7 - DNA, 21 bp, Artificial Sequence
  Description of Artificial Sequence synthetic oligonucleotide
catcgcacaaaccgccgtcat

SEQ ID NO: 8 - DNA, 21 bp, Artificial Sequence
  Description of Artificial Sequence synthetic oligonucleotide
acgcaggaacgccgggatatc

SEQ ID NO: 9 - DNA, 21 bp, Artificial Sequence
  Description of Artificial Sequence synthetic oligonucleotide
catcgccgcttccacactgct

SEQ ID NO: 10 - DNA, 21 bp, Artificial Sequence
  Description of Artificial Sequence synthetic oligonucleotide
gctgaggatggcgtaggcgat

SEQ ID NO: 11 - DNA, 21 bp, Artificial Sequence
  Description of Artificial Sequence synthetic oligonucleotide
tcaccacgtcgaacgtcggtg

SEQ ID NO: 12 - DNA, 21 bp, Artificial Sequence
  Description of Artificial Sequence synthetic oligonucleotide
ctccagcaggatgcgcaacat

SEQ ID NO: 13 - DNA, 21 bp, Artificial Sequence
  Description of Artificial Sequence synthetic oligonucleotide
aagtccggcgcgatggtcctg

SEQ ID NO: 14 - DNA, 21 bp, Artificial Sequence
  Description of Artificial Sequence synthetic oligonucleotide
gccaggatcgccagcaccagt

SEQ ID NO: 15 - DNA, 21 bp, Artificial Sequence
  Description of Artificial Sequence synthetic oligonucleotide
gcagagcggcaagatgatcgt

SEQ ID NO: 16 - DNA, 21 bp, Artificial Sequence
  Description of Artificial Sequence synthetic oligonucleotide
cttgggttcgtcgctgctgta

SEQ ID NO: 17 - DNA, 21 bp, Artificial Sequence
  Description of Artificial Sequence synthetic oligonucleotide
tggcgtactgctccgtcatca

SEQ ID NO: 18 - DNA, 21 bp, Artificial Sequence
  Description of Artificial Sequence synthetic oligonucleotide
ttggggtaacgcaggtcgatc

SEQ ID NO: 19 - DNA, 21 bp, Artificial Sequence
  Description of Artificial Sequence synthetic oligonucleotide
gccaccgcccagagcaactac

SEQ ID NO: 20 - DNA, 21 bp, Artificial Sequence
  Description of Artificial Sequence synthetic oligonucleotide
ctggctctgcagcaggctgac

SEQ ID NO: 21 - DNA, 21 bp, Artificial Sequence
  Description of Artificial Sequence synthetic oligonucleotide
gctcgagtcgacaggtctatt

SEQ ID NO: 22 - DNA, 21 bp, Artificial Sequence
  Description of Artificial Sequence synthetic oligonucleotide
gcgcaaggaaaagcagtatca

SEQ ID NO: 23 - DNA, 21 bp, Artificial Sequence
  Description of Artificial Sequence synthetic oligonucleotide
caccgtcaccctggatgctgt

SEQ ID NO: 24 - DNA, 21 bp, Artificial Sequence
  Description of Artificial Sequence synthetic oligonucleotide
ccatacccacgccgaaacaag